(19) United States
(12) Patent Application Publication
(10) Pub. No.: US 2024/0358982 A1
(43) Pub. Date: Oct. 31, 2024

(54) METHOD FOR ASSESSING A RISK OF RUPTURE OF A HOLLOW ORGAN, DATA PROCESSING APPARATUS, IMAGING SYSTEM, AND COMPUTER PROGRAM PRODUCT

(71) Applicant: Siemens Healthineers AG, Forchheim (DE)

(72) Inventors: Annette Birkhold, Stuttgart (DE); Philipp Roser, Erlangen (DE)

(21) Appl. No.: 18/635,445

(22) Filed: Apr. 15, 2024

(30) Foreign Application Priority Data
Apr. 27, 2023 (DE) 10 2023 203 919.2

(51) Int. Cl. A61M 25/10; A61B 6/00; A61B 6/50; A61M 25/00

(52) U.S. Cl. CPC A61M 25/10187; A61B 6/504; A61B 6/5205; A61B 6/5217; A61M 2025/0002; A61M 2205/3306

(57) ABSTRACT

To assess a risk of rupture of a hollow organ, at least one first image is obtained that maps a first expansion state of a balloon catheter disposed in the hollow organ. Based on the at least one first image, a balloon size and an internal balloon pressure of the balloon catheter in the first expansion state are determined. At least one second image is obtained that maps a second expansion state of the balloon catheter disposed in the hollow organ. Based on the at least one second image, the balloon size and the internal balloon pressure in the second expansion state are determined. Based on the internal balloon pressures in the first and second expansion states and the balloon sizes in the first and second expansion states, information is generated for assessing a risk of rupture of the hollow organ.

The present patent document claims the benefit of German Patent Application No. 10 2023 203 919.2, filed Apr. 27, 2023, which is hereby incorporated by reference in its entirety.

### TECHNICAL FIELD

The present disclosure relates to a method for assessing a risk of rupture of a hollow organ during balloon dilation. The disclosure further relates to a data processing apparatus for carrying out such a method, an imaging system including such a data processing apparatus, and a computer program product for carrying out a corresponding method.

### BACKGROUND

To perform balloon dilation, a balloon catheter is inserted into a hollow organ, in particular a vessel, (e.g., a blood vessel), and is selectively expanded by introducing a fluid, (e.g., air or a liquid), into the interior of the balloon catheter. This also widens out the hollow organ. In balloon pulmonary angioplasty (BPA), for example, balloon dilation is used to widen stenosed vessels in a patient's lungs. As hollow organs may only be stretched to a limited extent, there is a potential risk of the vessel rupturing during balloon dilation. Ruptures may represent significant complications in such interventions.

### SUMMARY AND DESCRIPTION

The object of the present disclosure is to assess or monitor the risk of rupture of a hollow organ during balloon dilation.

The scope of the present disclosure is defined solely by the appended claims and is not affected to any degree by the statements within this summary. The present embodiments may obviate one or more of the drawbacks or limitations in the related art.

The disclosure is based on the concept of determining, on the one hand, the balloon size of the balloon catheter and, on the other, the internal balloon pressure in the balloon catheter in two different expansion states of the balloon catheter. Depending on the parameters determined in this way, information is generated for assessing a risk of rupture of the hollow organ.

According to one aspect, a method is provided for assessing a risk of rupture of a hollow organ during balloon dilation. At least one first image is obtained, in particular by at least one computing unit, which maps a balloon catheter disposed in the hollow organ in a first expansion state of the balloon catheter and thus in particular of the hollow organ. Based on the at least one first image, the balloon size of the balloon catheter in the first expansion state is determined, in particular automatically by the at least one computing unit. The internal balloon pressure is determined in the first expansion state, in particular by the at least one computing unit. At least one second image is obtained, in particular by the at least one computing unit, which maps the balloon catheter disposed in the hollow organ in a second expansion state that is different in particular from the first expansion state of the balloon catheter or rather of the hollow organ. Based on the at least one second image, the balloon size in the second expansion state is determined automatically, in particular by the at least one computing unit, the balloon size in the second expansion state being different from the balloon size in the first expansion state, in particular being greater than the latter. The internal balloon pressure in the second expansion state is determined, in particular by the at least one computing unit.

Information for assessing a risk of rupture of the hollow organ is generated, in particular automatically by the at least one computing unit. The information is generated based on the internal balloon pressure in the first expansion state, the internal balloon pressure in the second expansion state, the balloon size in the first expansion state, and the balloon size in the second expansion state.

In various embodiments, the method may also be referred to as a method for assisting in the assessment of the risk of rupture, (e.g., by a user and/or automatically by the at least one computing unit). A further embodiment of the method proceeds directly from each embodiment of the method described here and in the following in that it incorporates the corresponding acts of generating the at least one first image, in particular by an imaging modality, and generating the at least one second image, in particular by the imaging modality.

The imaging modality may be an X-ray based imaging modality, for example, an X-ray based angiography system. However, in other embodiments, the imaging modality may also be a computed tomography system, an ultrasound-based imaging modality, or a magnetic resonance-based imaging modality, for example, a magnetic resonance-based angiography system.

The at least one first image and the at least one second image then correspond in particular to an image within the meaning of the respective imaging modality. In the example of an X-ray-based imaging modality, the images may each be X-ray projection images. In the case of a computed tomography system, the at least one image may be a three-dimensional reconstruction or a partial reconstruction from corresponding projection data.

The at least one first image and the at least one second image are acquired at different times or during different time periods. For example, all the images of the at least one first image may be generated at a first point in time or during a first time period and all images of the at least one second image may be generated at a second point in time or during a second time period. In particular, the second point in time is after the first point in time or the second time period is after the first time period. The first expansion state is present at the first point in time or during the entire first time period. The second expansion state is present at the second point in time or during the entire second time period.

Between the first and the second point in time or between the first time period and the second time period, the balloon catheter and thus the hollow organ transition from the first expansion state to the second expansion state. For this purpose, a quantity of fluid, in particular a gas or a liquid, (e.g., air, water, an aqueous solution or another solution), may be changed, (e.g., increased), in the interior of the balloon catheter. This may be done manually, for example, by pumping the fluid into the interior of the balloon catheter or by an automatic pumping apparatus. By introducing additional fluid into the interior of the balloon catheter, the internal pressure in the balloon catheter is increased, resulting in expansion, also known as dilation, of the balloon catheter and thus of the hollow organ. In particular, the balloon catheter lies against corresponding inner walls of the hollow organ, for example, against the vascular walls.

In the various embodiments of the method, neither the insertion of the balloon catheter into the hollow organ nor other surgical interventions are included in the method. In particular, the transitioning of the balloon catheter from the first expansion state to the second expansion state is also not to be understood as part of the method. Rather, selected expansion states are mapped and analyzed as described.

In order to assess the risk of rupture of the hollow organ, the information for assessing the risk of rupture may be output to a user, for example, visually via a display device, and the user may use the displayed information to assess the risk.

Alternatively or in addition, the risk assessment information may also be further processed or evaluated automatically or partially automatically, in particular automatically by the at least one computing unit, in order to assess the risk of rupture.

Use is made of the fact that the internal balloon pressure and the balloon size are interrelated. Because the hollow organ opposes the expansion of the balloon catheter, this relationship is influenced by the degree of risk of rupture. This is due to the fact that, depending on the expansion state of the hollow organ, different anatomical structures, in particular tissue components of the hollow organ, oppose the expansion of the balloon catheter with a mechanical force.

In the case of relatively small expansions, elastin fibers in particular counteract the expansion of the balloon catheter. In this regime, for example, the expansion state of the hollow organ is not critical and there is no risk of rupture or rather the risk of rupture is comparatively low. With stronger expansions and correspondingly stronger force applied to the hollow organ, the corresponding elastin fibers may be destroyed or damaged. With greater expansions, it is mainly collagen fibers that counteract the expansion. With even greater expansions, the collagen fibers may be destroyed or damaged, ultimately resulting in rupture of the hollow organ. Accordingly, the collagen-dominated region is a critical region in which the risk of rupture is increased, particularly in comparison to the elastin-dominated region.

The disclosure now makes use of the fact that the relationship between the internal balloon pressure, which is approximately equal to the internal cavity pressure, and the balloon size, which is directly linked to the distension of the hollow organ, are inter-dependent to varying degrees, depending on the region in which the expansion is located, i.e., whether the restoring forces are mainly collagen-dominated or mainly elastin-dominated. By determining both the internal balloon pressure and the balloon size both in the first expansion state and in the second expansion state, conclusions may be drawn about the change in the internal balloon pressure due to change in the balloon size, generated in the form of information on the risk of rupture and this information may possibly be used further.

In particular, use is made of the fact that with comparatively small tissue distensions, e.g., correspondingly also with a small balloon size, the change in internal pressure of a healthy hollow organ, in particular a vessel, for a given change in expansion, (e.g., a change in balloon size), is lower than with strong tissue distensions, e.g., in the collagen-dominated region. In other words, if the internal balloon pressure is plotted as a function of the balloon size, the gradient of the resulting curve is lower for small balloon sizes than for large balloon sizes. The gradient may therefore be used to assess the acute risk.

For example, the information for assessing the risk of rupture includes the gradient or is calculated based on the gradient. In particular, the gradient may be calculated approximately as the quotient of the difference between the internal balloon pressure in the second and first expansion states and the difference between the balloon size in the second and first expansion states.

The balloon size may be determined or defined differently in different embodiments of the method. It is in particular a one-dimensional size of the balloon, (e.g., a length dimension such as a radius or a diameter), or a two-dimensional size of the balloon, (e.g., a cross-sectional area), or a three-dimensional size, (e.g., a volume of the balloon), wherein the balloon size changes as the internal pressure of the balloon changes.

For example, the balloon size may correspond to the volume, in particular the internal volume, of the balloon catheter. The geometric shape of the balloon catheter may also be used to determine characteristic dimensions of the balloon catheter as the balloon size, for example, the diameter of the balloon catheter. In particular, the balloon catheter may be approximately cylindrical in shape, i.e., have a predetermined extent in a predetermined longitudinal direction, which in the case of a vessel may correspond to the vessel direction, and an at least approximately and/or sectionally constant diameter along the longitudinal direction. The diameter may then be determined from an image acquired with a viewing direction perpendicular to the longitudinal direction of the balloon catheter or a viewing direction parallel to the longitudinal direction of the balloon catheter.

If necessary, images with both of the aforementioned viewing directions may also be used to determine a more precise value for the diameter. Two images with different viewing directions, both perpendicular to the longitudinal direction of the balloon catheter, may also be used to determine a more accurate value for the diameter. The two viewing directions may be perpendicular to each other, for example.

If the balloon catheter were a circular cylinder, the diameter together with the given longitudinal expansion of the balloon catheter would unambiguously determine its volume. In the case of an approximately cylindrical balloon catheter, this applies at least approximately. However, the relationships between pressure and diameter may also be applied approximately in this case and therefore utilized accordingly.

The internal balloon pressure may likewise be determined in different ways. On the one hand, in various embodiments the internal balloon pressure may be measured directly, (e.g., using a pressure sensor inside the balloon catheter). Alternatively or in addition, a correlation between balloon size and internal balloon pressure may be determined in advance by calibration. In particular, the relationship between balloon size and internal balloon pressure may be determined in a reference state, e.g., outside a hollow organ. Due to the counterforce exerted on the balloon catheter by the hollow organ, this relationship is different from the relationship during the method, e.g., when the balloon catheter is disposed in the hollow organ. Accordingly, a direct measurement of the internal pressure based on the balloon size may be performed once for calibration, or the volume of the introduced fluid in the balloon catheter may be determined and the balloon size may be determined based on the volume of the introduced fluid. In the variants mentioned, when carrying out the method, it is possible to determine the internal balloon pressure in each expansion state directly or indirectly via the at least one image.

The acts of obtaining or generating the at least one second image, determining the internal balloon pressure and the balloon size, and generating the information for assessing the risk of rupture may be repeated for one or more further expansion states. The acts may also be performed continuously or quasi-continuously. The expansion state may change from one repetition to the next, namely when the volume of the fluid introduced into the balloon catheter is changed, in particular increased. However, the expansion state may also at times remain constant if the volume of the fluid introduced into the balloon catheter remains unchanged.

Accordingly, the balloon size and the internal balloon pressure are determined repeatedly, continuously, or quasi-continuously and the information for assessing the risk of rupture is accordingly determined repeatedly or updated. Thus, the risk assessment may be performed repeatedly, continuously, or quasi-continuously during balloon dilation, so that the risk of an actual rupture is reduced.

According to at least one embodiment, in particular to generate the information for assessing the risk of rupture, a visual representation is generated and displayed on a display device. In the visual representation, the internal balloon pressure in the first expansion state is associated with the balloon size in the first expansion state and the internal balloon pressure in the second expansion state is associated with the balloon size in the second expansion state. The information for assessing the risk of rupture includes, in particular, the visual representation.

The visual representation may be a representation in a two-dimensional coordinate system, wherein one axis, (e.g., the abscissa), of the coordinate system corresponds to the balloon size and another axis, (e.g., the ordinate), corresponds to the internal balloon pressure. The associations of the internal balloon pressure with the balloon size in the first and second expansion state then correspond to points in the coordinate system.

A user may see from the position of the two points relative to one another how much the internal balloon pressure has changed due to the change in balloon size from the first to the second expansion state and may assess the risk of rupture based on the relationships described above.

In embodiments in which internal balloon pressure and balloon size are repeatedly determined as described above, the visual representation may include a corresponding curve or interpolated curve. This allows the functional relationship between balloon size and internal balloon pressure to be determined more reliably. The correlation is easier for the user to read and less weight is given to possible outliers or incorrect measurements.

According to at least one embodiment, in particular in order to generate the information for assessing the risk of rupture, a pressure difference between the internal balloon pressure in the first expansion state and the internal balloon pressure in the second expansion state is calculated, in particular by the at least one computing unit, a size difference between the balloon size in the first expansion state and the balloon size in the second expansion state is calculated, in particular by the at least one computing unit, and a risk value for the rupture is calculated based on a ratio of the pressure difference to the size difference, in particular by the at least one computing unit.

The risk value may be output to the user, for example, displayed to the user, and/or the risk value may be stored so that a user may retrieve the risk value if required. Alternatively or in addition, the risk value may be further processed, for example, compared with a limit value, in particular by the at least one computing unit. Depending on the result, appropriate action may be taken, for example, a warning may be issued.

The risk value may be a first risk value that is given by a quotient of the pressure difference and the size difference, e.g., the pressure difference divided by the size difference, or is proportional thereto, in particular directly proportional. The greater the first risk value, the greater the resistance of the hollow organ to the expansion of the balloon catheter. Therefore, the greater the first risk value, the greater the risk of rupture.

Alternatively, the risk value may be a second risk value which is given by a quotient of the size difference and the pressure difference, e.g., the size difference divided by the pressure difference, or is proportional thereto, in particular directly proportional. The greater the second risk value, the less resistance the hollow organ offers to the expansion of the balloon catheter. Therefore, the smaller the second risk value, the greater the risk of rupture. The second risk value may also be referred to as the compliance value.

According to at least one embodiment, the risk value is compared with a predetermined limit value, in particular by the at least one computing unit, and a warning message is generated and output, for example, visually and/or audibly, depending on the result of the comparison.

In this way, the user may be made aware of any increased risk of rupture at an early stage.

In one embodiment, the first risk value is compared with a first limit value. The warning message is generated and output if the first risk value is greater than the first limit value or if the first risk value is greater than or equal to the first limit value.

In one embodiment, the second risk value is compared with a second limit value. The warning message is generated and output if the second risk value is less than the second limit value or if the second risk value is less than or equal to the second limit value.

According to at least one embodiment, a sensor signal is received, in particular by the at least one computing unit, from a pressure sensor disposed inside the balloon catheter. The internal balloon pressure in the first expansion state and/or the internal balloon pressure in the second expansion state are determined based on the sensor signal, in particular by the at least one computing unit.

In particular, the sensor signal is generated by the pressure sensor based on the internal balloon pressure and provided to the at least one computing unit. In this way, a particularly reliable determination of the internal balloon pressure may be achieved, thereby increasing the accuracy of the estimation of the risk of rupture.

The pressure sensor may be connected to the at least one computing unit for example via a feed line that is also used to introduce the fluid into the hollow organ, or via a separate feeder. Alternatively, the pressure sensor may communicate wirelessly with the at least one computing unit in order to provide the sensor signal.

In particular, the sensor signal is generated when the first expansion state is present and generated again when the second expansion state is present. The sensor signal may also be generated continuously and then evaluated when the first expansion state is present and generated again when the second expansion state is present.

According to at least one embodiment, the internal balloon pressure in the first expansion state is determined based on the balloon size in the first expansion state using a predetermined association rule, in particular by the at least one computing unit. Alternatively or in addition, the internal balloon pressure in the second expansion state is determined based on the balloon size in the second expansion state using the association rule, in particular by the at least one computing unit.

As mentioned above, the association rule may be created by calibration under known reference conditions. The association rule may associate the internal balloon pressure directly with a given balloon size or indirectly, e.g., by taking into account other input information in addition to the balloon size.

In this way, the internal balloon pressure may be evaluated without a dedicated pressure sensor. The association rule may correspond to a look-up table or similar.

According to at least one embodiment, a first amount of fluid present inside the balloon catheter in the first expansion state is determined and the internal balloon pressure in the first expansion state is associated with the first amount of fluid and the balloon size in the first expansion state in accordance with the association rule.

According to at least one embodiment, a second quantity of fluid present inside the balloon catheter in the second expansion state is determined and the internal balloon pressure in the second expansion state is associated with the second quantity of fluid and the balloon size in the second expansion state in accordance with the association rule.

The amount of fluid may correspond to a total volume of fluid introduced into the balloon catheter. For example, the user may manually introduce the fluid into the balloon interior, in particular by actuating a pump mechanism. The number and/or the stroke of the pumping operations may be determined manually or automatically, and thus the amount of fluid present inside the balloon may be evaluated. Alternatively, the flow rate of the fluid may be measured, in particular also in the case of automatic introduction of the fluid into the balloon interior, in order to determine the amount of fluid for the first and second expansion state.

According to at least one embodiment, a first nominal balloon size of the balloon catheter is determined based on the first fluid quantity, a first further size difference between the first nominal balloon size and the balloon size in the first expansion state is determined, and the internal balloon pressure in the first expansion state is determined based on the first further size difference in accordance with the association rule.

According to at least one embodiment, a second nominal balloon size of the balloon catheter is determined based on the second fluid quantity, a second further size difference between the second nominal balloon size and the balloon size in the second expansion state is determined, and the internal balloon pressure in the second expansion state is determined based on the second further size difference in accordance with the association rule.

The first nominal balloon size or the second nominal balloon size correspond in particular to balloon sizes of the balloon catheter under ideal conditions or predefined standard conditions, for example under ambient pressure, e.g., when the balloon catheter is not disposed in the hollow organ.

The corresponding calibration may be used to determine how large the respective expansion of the balloon catheter should be if it were not located in a hollow organ and a particular amount of fluid were introduced. Due to the resistance of the hollow organ to the expansion of the balloon catheter, however, the size of the balloon catheter is smaller than the nominal balloon size. The greater the difference, the greater the difference between the actual internal balloon pressure and the nominally expected internal balloon pressure, so that the actual internal balloon pressure in the first and second expansion state may be determined in accordance with the association rule.

According to at least one embodiment, the balloon size corresponds to a lateral expansion, e.g., a lateral diameter or radius of the balloon catheter perpendicular to the longitudinal axis of the balloon catheter.

In particular, the balloon catheter in this case is an approximately or essentially cylindrical, in particular circular-cylindrical, balloon catheter. The balloon size in the first expansion state and the balloon size in the second expansion state are each determined in particular as the corresponding lateral expansion of the balloon catheter.

This provides a particularly simple determining the characteristic balloon size.

Alternatively, the balloon size is in each case a corresponding volume, in particular the internal volume, of the balloon catheter. Accordingly, the balloon size in the first expansion state and the balloon size in the second expansion state are each determined as corresponding volumes of the balloon catheter.

In this way, a more accurate assessment of the risk of rupture may be achieved, because deviations from the assumed cylindrical shape of the balloon catheter may also be taken into account or rather the risk may also be assessed for more complex geometries of the balloon catheter or other geometries.

In embodiments in which the balloon size is the volume of the balloon catheter, the at least one first image may include two or more first images and the at least one second image may include two or more second images. The first balloon size is determined based on the two or more first images, and the second balloon size is determined based on the two or more second images.

According to at least one embodiment, the at least one first image includes two or more first images, wherein respective viewing directions, also referred to as acquisition directions, of the two or more first images are different from one another.

In other words, all of the images of the two or more first images are generated with different viewing or acquisition directions in each case.

According to at least one embodiment, the at least one second image includes two or more second images, wherein respective viewing directions of the two or more second images are different from one another.

In other words, all of the images of the two or more second images are generated with different viewing or acquisition directions in each case.

For example, the viewing direction may be a direction perpendicular to an X-ray projection surface if the imaging modality is an X-ray based imaging modality, or perpendicular to an MR imaging plane if the imaging modality is an MR system. In the case of an ultrasound-based imaging modality, the viewing direction may be parallel to a main propagation direction of the ultrasound waves used for examination/imaging.

From each first image of the two or more first images, a corresponding characteristic expansion of the balloon catheter in the first expansion state may be determined. The volume of the balloon catheter in the first expansion state may then be determined from the characteristic expansions.

The different viewing directions of the two or more first images include, for example, a viewing direction perpendicular to the longitudinal axis of the balloon catheter and a viewing direction parallel to the longitudinal axis of the balloon catheter.

From each second image of the two or more second images, a corresponding characteristic expansion of the balloon catheter in the second expansion state may be determined. The volume of the balloon catheter in the second expansion state may then be determined from the characteristic expansions.

The different viewing directions of the two or more second images include, for example, the viewing direction perpendicular to the longitudinal axis of the balloon catheter and the viewing direction parallel to the longitudinal axis of the balloon catheter.

The at least one first image or the at least one second image may thus be generated, for example, by biplanar or multiplanar X-ray imaging.

In the case of a computed tomography apparatus as the imaging modality, a three-dimensional reconstruction or partial reconstruction of the balloon catheter may be generated from the respective acquired images and the volume of the balloon catheter may be determined in each case based on the reconstruction or partial reconstruction.

For use cases or use situations that may arise in the method, and which are not explicitly described herein, it may be provided that, according to the method, an error message and/or a request to enter user feedback is output and/or a default setting and/or a predetermined initial state is selected.

According to a further aspect, a data processing apparatus having at least one computing unit is specified. The at least one computing unit is designed to carry out a method for assessing the risk of rupture of a hollow organ during balloon dilation.

In particular, a computing unit may be understood as meaning a data processing apparatus which contains a processing circuit. The computing unit may therefore in particular process data for performing computing operations. This may also include operations to perform indexed accesses to a data structure, for example a look-up table (LUT).

In particular, the computing unit may include one or more computers, one or more microcontrollers, and/or one or more integrated circuits, (e.g., one or more application-specific integrated circuits (ASICs), one or more field-programmable gate arrays (FPGAs), and/or one or more systems-on-a-chip (SoCs)). The computing unit may also include one or more processors, (e.g., one or more microprocessors), one or more central processing units (CPUs), one or more graphics processing units (GPUs), and/or one or more signal processors, (e.g., one or more digital signal processors (DSPs)). The computing unit may also include a physical or virtual network of computers or other such units.

In various embodiments, the computing unit includes one or more hardware and/or software interfaces and/or one or more memory units.

A memory unit may be implemented as volatile data memory, for example as dynamic random-access memory (DRAM) or static random-access memory (SRAM), or as non-volatile data memory, for example as read-only memory (ROM), as programmable read-only memory (PROM) or as erasable programmable read-only memory (EPROM), as electrically erasable programmable read-only memory (EEPROM), as flash memory or flash EEPROM, as ferroelectric random-access memory (FRAM), as magnetoresistive random-access memory (MRAM) or as phase-change random-access memory (PCRAM).

According to a further aspect, an imaging system for monitoring balloon dilation of a hollow organ is specified. The imaging system has an imaging modality and a data processing apparatus. The imaging modality is designed to generate the at least one first image and to generate the at least one second image.

According to at least one embodiment of the imaging system, the imaging system incorporates the balloon catheter.

According to at least one embodiment, the imaging modality is designed as an X-ray based imaging modality, for example as an X-ray based angiography system or as a computed tomography system, or the imaging modality is designed as an MR imaging modality, for example as a magnetic resonance based angiography system, or as an ultrasound based imaging system.

Further embodiments of the imaging system proceed directly from the various embodiments of the method and vice versa. In particular, individual features and corresponding explanations as well as advantages relating to the various embodiments of the method may be applied analogously to corresponding embodiments of the imaging system. In particular, the imaging system is configured or programmed to carry out a method. In particular, the imaging system carries out the method.

According to a further aspect, a computer program including instructions is provided. When the instructions are executed by a data processing apparatus, in particular the at least one computing unit, the instructions cause the data processing apparatus to carry out a method for assessing a risk of rupture of a hollow organ. The method does not include the acts of generating the at least one first image and the at least one second image by the imaging modality.

According to a further aspect, a further computer program including further instructions is provided. When the further instructions are executed by an imaging system, in particular by the data processing apparatus of the imaging system, the further instructions cause the imaging system to carry out a method for assessing a risk of rupture of a hollow organ. The method includes the acts of generating the at least one first image and the at least one second image by the imaging modality.

The instructions and/or the further instructions may be in the form of program code. The program code may be provided as binary code or assembler and/or as source code of a programming language, (e.g., C), and/or as a program script, (e.g., Python).

According to a further aspect, a computer-readable storage medium is provided which stores a computer program and/or a further computer program.

The computer program, the further computer program, and the computer-readable storage medium may each be understood as being a computer program product containing the instructions and/or the further instructions.

Further features of the disclosure emerge from the claims, the figures, and the description of the figures. The features and combinations of features mentioned above in the description and the features and combinations of features mentioned below in the description of the figures and/or shown in the figures may be within the scope the disclosure not only in the combination indicated in each case, but also in other combinations. In particular, embodiments and combinations of features may also be within the scope of the disclosure which do not have all the features of an originally formulated claim. In addition, the disclosure may include embodiments and combinations of features which go beyond or deviate from the combinations of features set forth in the references of the claims.

### BRIEF DESCRIPTION OF THE DRAWINGS

The disclosure is now explained in more detail with reference to specific embodiments and associated schematic drawings. In the figures, identical or functionally identical elements may be provided with the same reference characters. The description of identical or functionally identical elements may not necessarily be repeated with respect to different figures, wherein:

FIG. 1 schematically illustrates an embodiment of an imaging system for monitoring balloon dilation of a hollow organ.

FIG. 2 shows a schematic flowchart of an embodiment of a method for assessing a risk of rupture of a hollow organ during balloon dilation.

FIG. 3 schematically illustrates an example of a balloon catheter in two different expansion states.

FIG. 4 shows a visual representation of the internal balloon pressure of a balloon catheter based on the diameter of the balloon catheter for another embodiment of a method.

### DETAILED DESCRIPTION

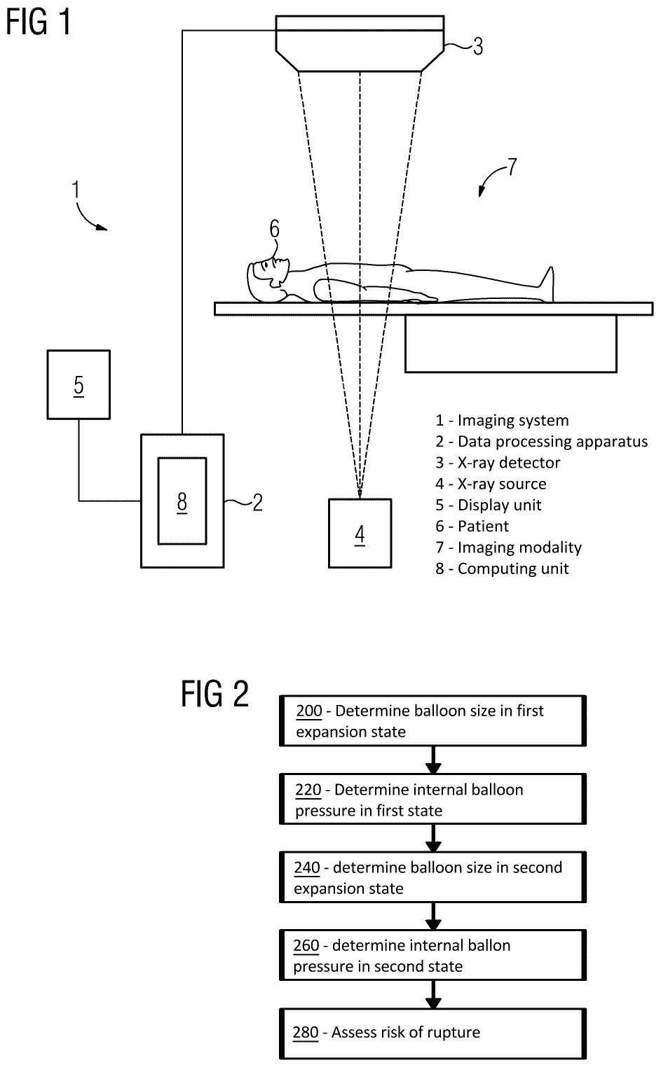

FIG. 1 schematically illustrates an embodiment of an imaging system 1 for monitoring balloon dilation of a hollow organ of a patient 6. The imaging system 1 has a data processing apparatus 2 that incorporates at least one computing unit 8. The imaging system 1 also has an imaging modality 7.

The imaging modality 7 may be designed, for example, as an X-ray-based imaging modality for angiography, in particular for balloon pulmonary angioplasty (BPA). The imaging modality 7 then has, for example, an X-ray source 4 and an X-ray detector 3, and the patient 6 may be placed in the beam path of the X-ray source 4 between the X-ray source 4 and the X-ray detector 3 in order to enable the balloon dilation to be monitored by X-ray imaging.

The imaging system 1 optionally has a display unit 5 on which the at least one computing unit 8 may display information for assessing a risk of rupture of the hollow organ.

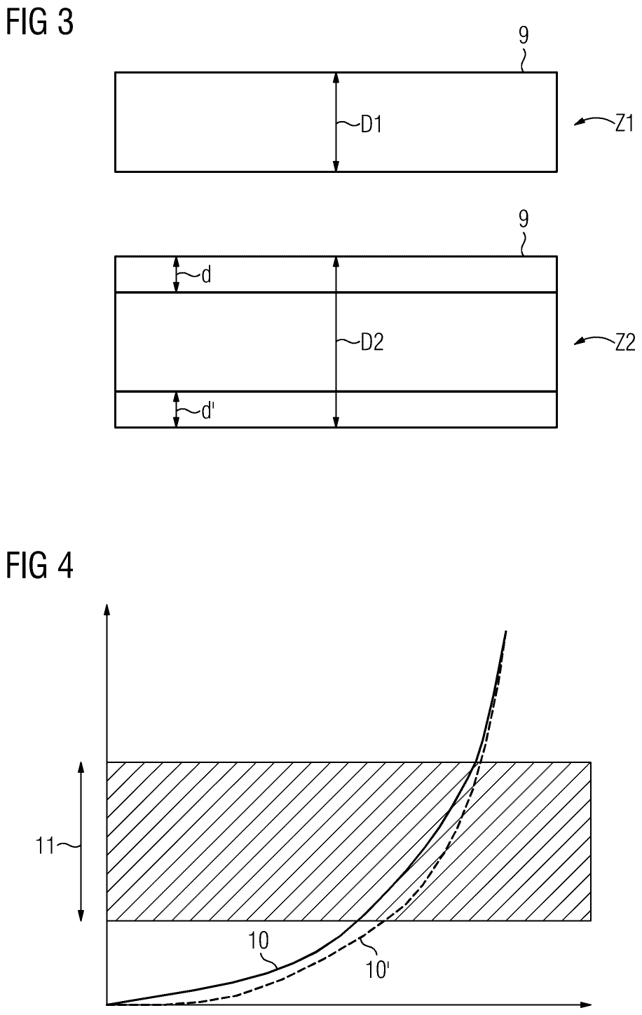

The imaging modality 7 is configured to generate at least one first image mapping a first expansion state Z1 of a balloon catheter 9 (see FIG. 3) disposed in the hollow organ which may be a pulmonary vessel, for example. The imaging modality 7 is further configured to generate at least one second image mapping a second expansion state Z2 of the balloon catheter 9 disposed in the hollow organ.

The at least one computing unit 8 is configured to receive the at least one first image and the at least one second image and, based thereon, to perform a method for assessing the risk of rupture of the hollow organ.

A schematic flowchart of such a method is shown in FIG. 2. In act 200, the at least one computing unit 8 determines the balloon size of the balloon catheter 9 in the first expansion state Z1 based on the at least one first image, for example as the first diameter D1 of the balloon catheter 9.

In act 220, the at least one computing unit 8 determines the internal balloon pressure of the balloon catheter 9 in the first expansion state Z1. For this purpose, the at least one computing unit 8 may receive and evaluate a sensor signal from a pressure sensor (not shown) disposed inside the balloon catheter 9. Alternatively, the at least one computing unit 8 may estimate the internal balloon pressure depending on the balloon size of the balloon catheter 9 in the first expansion state Z1 using a predetermined association rule.

In act 240, the at least one computing unit 8 determines the balloon size of the balloon catheter 9 in the second expansion state Z2 based on the at least one second image, for example, as the second diameter D2 of the balloon catheter 9, which differs from the first diameter D1 by a diameter difference d, d′.

In act 260, the at least one computing unit 8 determines the internal balloon pressure of the balloon catheter 9 in the second expansion state Z1. For this purpose, the at least one computing unit 8 may evaluate the sensor signal. Alternatively, the at least one computing unit 8 may estimate the internal balloon pressure based on the balloon size of the balloon catheter 9 in the second expansion state Z2 using a predetermined association rule.

In act S280, the at least one computing unit 8 generates information for assessing the risk of rupture of the hollow organ based on the internal balloon pressure in the first expansion state Z1, the internal balloon pressure in the second expansion state Z2, the balloon size in the first expansion state Z1, and the balloon size in the second expansion state Z2.

For example, the at least one computing unit 8 may calculate a compliance value C as follows:

$$C = \frac{D 2 - D 1}{D 1} \frac{1}{P 2 - P 1} ,$$

where P1 is the internal balloon pressure in the first expansion state Z1, and P2 is the internal balloon pressure in the second expansion state Z2.

The at least one computing unit 8 may compare the compliance value C with a predetermined limit value and generate a warning message and output it to the user if the compliance value C is less than the limit value.

Alternatively or in addition, the at least one computing unit 8 may generate a visual representation based on the determined internal balloon pressures and the determined balloon sizes and display it on the display device 5. An example is shown schematically in FIG. 4.

FIG. 4 shows a two-dimensional coordinate system, wherein the balloon size, in particular the diameter of the balloon, is plotted on the abscissa and the internal balloon pressure is plotted on the ordinate. If the diameter of the balloon catheter 9 is increased by introducing fluid into the interior of the balloon catheter 9, this results in a pressure curve 10. If the diameter is reduced by removing fluid from the interior of the balloon catheter 9, this results in another pressure curve 10′. The shaded area 11 corresponds to a physiological range for a blood vessel, for example from 80 to 120 mmHg.

At pressures above this physiological range 11, the behavior of the hollow organ may be collagen-dominated. At pressures below the physiological range 11, the behavior of the hollow organ may be elastin-dominated. As may be seen from the pressure curves 10, 10′, the gradient of the pressure based on the diameter is higher in the collagen-dominated range than in the elastin-dominated range. By displaying the pressure curve as functions of the diameter, the user may therefore estimate in which region the hollow organ is located and how high the risk of rupture is. In some embodiments, a reference curve or one or more thresholds may also be displayed in the visual display to provide better guidance and risk assessment.

As described, particularly with reference to the figures, the disclosure makes it possible to assess the risk of rupture of a hollow organ, for example a vessel, during balloon dilation, for example BPA.

In various embodiments, a balloon catheter, (e.g., an approximately circular cylindrical balloon catheter), is used for which it has been determined in advance, in particular by calibration, how the change in the diameter of the balloon catheter is related to a change in the internal pressure, for example when a known amount of fluid is introduced into the interior of the balloon catheter.

In some embodiments, the internal balloon pressure may be measured by a pressure sensor during balloon dilation.

In some embodiments, the balloon catheter may be detected and tracked during live imaging, such as live fluoroscopy, for example. Known segmentation methods may be used to identify the image region corresponding to the balloon catheter. Based on this, the balloon diameter and thus the diameter of the hollow organ may be estimated. If biplanar imaging is used, the accuracy of the estimate may be improved. Then, for example, a diagram may be created that shows the internal balloon pressure based on the diameter. Based on the gradient of the corresponding curve, for example, the effect of elastin fibers and the contribution of collagen fibers may be estimated and the risk of rupture assessed accordingly. If necessary, a threshold value for maximum possible dilation may be determined based on the curve, for example, in the form of a vessel compliance.

In some embodiments, the balloon volume may be estimated based on the segmented balloon area in the images. The expected balloon volume based on the inflation pressure may be compared with the actual balloon volume and, on the basis thereof, the volume-pressure relationship of the vessel walls may be assessed as a surrogate for the risk of rupture.

In some embodiments, a continuous comparison of the expected and actual balloon volume or balloon diameter may be used to detect non-physiological behavior of the vessel.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that changes and modifications may be made to the described embodiments. It is therefore intended that the foregoing description be regarded as illustrative rather than limiting, and that it be understood that all equivalents and/or combinations of embodiments are intended to be included in this description.

1. A method for assessing a risk of rupture of a hollow organ during balloon dilation, the method comprising:
obtaining at least one first image that maps a first expansion state of a balloon catheter disposed in the hollow organ;
determining a balloon size of the balloon catheter in the first expansion state based on the at least one first image;
determining an internal balloon pressure of the balloon catheter in the first expansion state;
obtaining at least one second image that maps a second expansion state of the balloon catheter disposed in the hollow organ;
determining the balloon size in the second expansion state based on the at least one second image;
determining the internal balloon pressure in the second expansion state; and
generating information for assessing a risk of rupture of the hollow organ based on the internal balloon pressure in the first expansion state, the internal balloon pressure in the second expansion state, the balloon size in the first expansion state, and the balloon size in the second expansion state.

2. The method of claim 1, wherein a visual representation is generated and displayed on a display device, and
wherein, in the visual representation, the internal balloon pressure in the first expansion state is associated with the balloon size in the first expansion state and the internal balloon pressure in the second expansion state is associated with the balloon size in the second expansion state.

3. The method of claim 1, further comprising:
calculating a pressure difference between the internal balloon pressure in the first expansion state and the internal balloon pressure in the second expansion state;
calculating a size difference between the balloon size in the first expansion state and the balloon size in the second expansion state; and
calculating a risk value for the rupture based on a ratio of the pressure difference to the size difference.

4. The method of claim 3, further comprising:
comparing the risk value with a predetermined limit value; and
generating and outputting a warning message depending on a result of the comparison.

5. The method of claim 1, wherein a sensor signal is received from a pressure sensor disposed inside the balloon catheter, and
wherein the internal balloon pressure in the first expansion state and/or the internal balloon pressure in the second expansion state are determined based on the sensor signal.

6. The method of claim 1, wherein the internal balloon pressure in the first expansion state is determined based on the balloon size in the first expansion state using a predetermined association rule, and/or
wherein the internal balloon pressure in the second expansion state is determined based on the balloon size in the second expansion state using the association rule.

7. The method of claim 6, wherein a first quantity of fluid present inside the balloon catheter in the first expansion state is determined and the internal balloon pressure in the first expansion state is associated with the first quantity of fluid and the balloon size in the first expansion state in accordance with the association rule, and/or
wherein a second quantity of fluid present inside the balloon catheter in the second expansion state is determined and the internal balloon pressure in the second expansion state is associated with the second quantity of fluid and the balloon size in the second expansion state in accordance with the association rule.

8. The method of claim 7, wherein a first nominal balloon size of the balloon catheter is determined based on the first fluid quantity, wherein a first further size difference between the first nominal balloon size and the balloon size in the first expansion state is determined, and wherein the internal balloon pressure in the first expansion state is determined based on the first further size difference in accordance with the association rule, and/or
wherein a second nominal balloon size of the balloon catheter is determined based on the second fluid quantity, wherein a second further size difference between the second nominal balloon size and the balloon size in the second expansion state is determined, and wherein the internal balloon pressure in the second expansion state is determined based on the second further size difference in accordance with the association rule.

9. The method of claim 1, wherein the balloon size in the first expansion state and the balloon size in the second expansion state are each determined as a corresponding lateral expansion perpendicular to a longitudinal axis of the balloon catheter.

10. The method of claim 1, wherein the balloon size in the first expansion state and the balloon size in the second expansion state are each determined as the corresponding volume of the balloon catheter.

11. The method of claim 1, wherein the at least one first image comprises two or more first images, wherein respective viewing directions of the two or more first images differ from each other, and/or
wherein the at least one second image comprises two or more second images, wherein respective viewing directions of the two or more second images differ from each other.

12. A data processing apparatus comprising:
at least one computing unit configured to:
obtain at least one first image that maps a first expansion state of a balloon catheter disposed in the hollow organ;
determine a balloon size of the balloon catheter in the first expansion state based on the at least one first image;
determine an internal balloon pressure of the balloon catheter in the first expansion state;
obtain at least one second image that maps a second expansion state of the balloon catheter disposed in the hollow organ;
determine the balloon size in the second expansion state based on the at least one second image;
determine the internal balloon pressure in the second expansion state; and
generate information for assessing a risk of rupture of the hollow organ based on the internal balloon pressure in the first expansion state, the internal balloon pressure in the second expansion state, the balloon size in the first expansion state, and the balloon size in the second expansion state.

13. An imaging system for monitoring balloon dilation of a hollow organ, the imaging system comprising:
an imaging modality configured to generate at least one first image and to generate at least one second image; and
a data processing apparatus having an at least one computing unit configured to:
obtain the at least one first image that maps a first expansion state of a balloon catheter disposed in the hollow organ;
determine a balloon size of the balloon catheter in the first expansion state based on the at least one first image;
determine an internal balloon pressure of the balloon catheter in the first expansion state;
obtain the at least one second image that maps a second expansion state of the balloon catheter disposed in the hollow organ;
determine the balloon size in the second expansion state based on the at least one second image;
determine the internal balloon pressure in the second expansion state; and
generate information for assessing a risk of rupture of the hollow organ based on the internal balloon pressure in the first expansion state, the internal balloon pressure in the second expansion state, the balloon size in the first expansion state, and the balloon size in the second expansion state.

14. The imaging system of claim 13, wherein the imaging modality is an X-ray based angiography system, a computed tomography system, a magnetic resonance based angiography system, or an ultrasound based imaging system.
